(19) United States
(12) Patent Application Publication
(10) Pub. No.: US 2018/0328656 A1
(43) Pub. Date: Nov. 15, 2018

(54) METHODS FOR RECOVERING ALKENES FROM PROCESS GAS STREAMS

(71) Applicant: Linde Aktiengesellschaft, Munich (DE)

(72) Inventor: Nicole RUMORE, Monmouth Junction, NJ (US)

(21) Appl. No.: 15/591,860

(22) Filed: May 10, 2017

(51) Int. Cl. F25J 3/06; C07C 7/09; F25J 3/08

(52) U.S. Cl. CPC F25J 3/064; C07C 7/09; F25J 3/066; F25J 3/0655; F25J 2210/02; F25J 2215/62; F25J 2215/64; F25J 2245/02; F25J 2270/904; F25J 3/08

(57) ABSTRACT

Methods and systems for recovering alkenes (e.g. ethylene, propylene) from process gas streams, including multi-step condensing of the process gas stream, are provided herein.

### FIELD OF THE INVENTION

This invention relates to methods for recovering alkene gas and removing impurities from various process gas streams.

### BACKGROUND OF THE INVENTION

Valuable hydrocarbons, such as lower alkenes, may be present in a multitude of process gas streams at varying concentrations and typically amongst other components. For example, ethylene and/or propylene may be present in cracked gas streams from hydrocarbon crackers, which also may contain nitrogen, methane, ethane, propane, hydrogen, other higher hydrocarbons and other impurities. Additionally, during polymerization processes, unreacted ethylene or propylene monomer may be present in streams along with inert purge gases, such as nitrogen. Typically, such streams containing alkene process gas (e.g., ethylene, propylene) are removed from the system via flaring or with a thermal oxidizer. Prior to flaring or thermal oxidizing, recovery processes may be employed for recovering as much of the alkene as possible so it is not lost when flared or oxidized. Examples of existing recovery processes include compression and condensation systems as well as use of pressure swing adsorption systems and/or membranes. However, existing recovery processes may still be limited in the amount of alkene recovery and impurity removal and even further processing may be required at additional costs to recover further amounts of alkene. Moreover, process gas streams comprising alkene may have higher temperatures; thus, requiring additional energy and resources, such as liquid nitrogen, to sufficiently lower the temperature of the process gas stream such that it may be suitably utilized in a condensation system.

Thus, a need remains for improved and more effective methods which utilize heat integration for recovery of alkenes and removal of impurities from process gas streams.

### SUMMARY OF THE INVENTION

It has been found that impurities from process gas streams may be sufficiently removed and alkenes may be sufficiently recovered by performing a combination of process steps including cooling a process gas stream to remove a portion of the impurities via a condensate stream followed by further cooling of the process gas stream for alkene recovery. Furthermore, streams produced during the process may be advantageously recycled and utilized during the process and provide sufficient cooling of the process gas streams.

Thus, in one aspect, this disclosure relates to a method for recovering C₂-C₄ alkene from a process gas stream comprising: a first condensation cycle comprising: cooling the process gas stream comprising C₂-C₄ alkene and impurities in a first condenser vessel with a first cooling medium under suitable conditions to produce a first condensate comprising at least a portion of the impurities and a first cooled process gas stream comprising C₂-C₄ alkene having a temperature of less than or equal to −10° C.; collecting the first condensate in a first condensate tank; cooling the first cooled process gas stream comprising C₂-C₄ alkene in a second condenser vessel with a second cooling medium under suitable conditions to produce a second condensate comprising C₂-C₄ alkene and a first vent gas stream; and collecting the second condensate in a second condensate tank; and a pressurization cycle comprising: heating the second condensate tank to produce pressurized liquid C₂-C₄ alkene; and optionally, recycling at least a portion of the pressurized liquid C₂-C₄ alkene to a second condensation cycle comprising the same steps as the first condensation cycle for use as the first cooling medium.

In still another aspect, this disclosure relates to a system for recovering C₂-C₄ alkene from a process gas stream comprising: a first process gas stream comprising C₂-C₄ alkene; a first cooling medium stream; a first condensate stream comprising at least a portion of the impurities; a first cooled process gas stream having a temperature of less than or equal to −10° C.; a first vent gas stream; a second cooling medium stream; a second condensate stream comprising C₂-C₄ alkene; a second process gas stream comprising C₂-C₄ alkene; a third cooling medium stream; a third condensate stream comprising at least a portion of the impurities; a second cooled process gas stream having a temperature of less than or equal to −10° C.; a second vent gas stream; a fourth cooling medium stream; a fourth condensate stream comprising C₂-C₄ alkene; a first pressurized liquid C₂-C₄ alkene stream; a first C₂-C₄ alkene vent gas stream; a first condenser vessel operated under suitable conditions to produce the first condensate stream comprising at least a portion of the impurities and the first cooled process gas stream, wherein the first condenser vessel comprises: a first heat exchanger; a first inlet for providing the first process gas stream; a second inlet for providing the first cooling medium; a first outlet for removal of a first spent cooling medium; a second outlet for removal of the first cooled process gas stream; and a third outlet for removal of the first condensate stream; a first condensate tank comprising: a third inlet for providing the first condensate stream; a second condenser vessel operated under suitable conditions to produce the second condensate stream comprising C₂-C₄ alkene and the first vent gas stream, wherein the second condenser vessel comprises: a second heat exchanger; a fourth inlet for providing the first cooled process gas stream; a fifth inlet for providing the second cooling medium; a fourth outlet for removal of a second spent cooling medium; a fifth outlet for removal of the first vent gas stream; and a sixth outlet for removal of the second condensate stream; a second condensate tank comprising: a sixth inlet for providing the second condensate stream; a seventh outlet for removal of the first pressurized liquid C₂-C₄ alkene stream; and an eighth outlet for removal the first C₂-C₄ alkene vent gas stream; a first means for providing heat to the second condensate tank; a third condenser vessel operated under suitable conditions to produce the third condensate stream comprising at least a portion of the impurities and the second cooled process gas stream, wherein the third condenser vessel comprises: a third heat exchanger; a seventh inlet for providing the second process gas stream; an eighth inlet for providing the third cooling medium; a ninth outlet for removal of a third spent cooling medium; a tenth outlet for removal of the second cooled process gas stream; and an eleventh outlet for removal of the third condensate stream; a third condensate tank comprising: a ninth inlet for providing the third condensate stream; a fourth condenser vessel operated under suitable conditions to produce the fourth condensate stream comprising C₂-C₄ alkene and the second vent gas stream, wherein the fourth condenser vessel comprises: a fourth heat exchanger; a tenth inlet for providing the second cooled process gas stream; an eleventh inlet for providing the fourth cooling medium; a twelfth outlet for removal of a fourth spent cooling medium; eleventh thirteenth outlet for removal of the second vent gas stream; and a fourteenth outlet for removal of the fourth condensate stream; and a fourth condensate tank comprising: a twelfth inlet for providing the fourth condensate stream.

Other embodiments, including particular aspects of the embodiments summarized above, will be evident from the detailed description that follows.

### BRIEF DESCRIPTION OF THE DRAWINGS

The drawings described herein are for illustrative purposes only of selected embodiments and not all possible implementations, and are not intended to limit the scope of the present disclosure.

FIG. 1 illustrates a sequence of cycles in two systems running in parallel for recovering alkene gas and removing impurities from various process gas streams.

FIG. 2 illustrates a schematic of a system for recovering alkenes from process gas streams according to certain aspects of the present disclosure.

FIG. 3 illustrates a schematic of a system for recovering alkenes from process gas streams according to certain alternative aspects of the present disclosure.

Corresponding reference numerals indicate corresponding parts throughout the several views of the drawings.

### DETAILED DESCRIPTION OF THE INVENTION

In various aspects of the invention, methods and systems for recovering alkenes from process gas streams are provided.

### I. Definitions

The term “and/or” as used in a phrase such as “A and/or B” herein is intended to include “A and B”, “A or B”, “A”, and “B”.

As used herein, and unless otherwise specified, the term “Ca” means hydrocarbon(s) having n carbon atom(s) per molecule, wherein n is a positive integer. As used herein, and unless otherwise specified, the term “hydrocarbon” means a class of compounds containing hydrogen bound to carbon, and encompasses (i) saturated hydrocarbon compounds, (ii) unsaturated hydrocarbon compounds, and (iii) mixtures of hydrocarbon compounds (saturated and/or unsaturated), including mixtures of hydrocarbon compounds having different values of n.

As used herein, the term “alkene,” alternatively referred to as “olefin,” refers to a branched or unbranched unsaturated hydrocarbon having one or more carbon-carbon double bonds. A simple alkene comprises the general formula CₙH₂ₙ, where n is 2 or greater. Examples of alkenes include, but are not limited to ethene, propene, butene, pentene, hexene and heptene. “Alkene” is intended to embrace all structural isomeric forms of an alkene. For example, butene encompasses but-1-ene, (Z)-but-2-ene, etc.

As used herein, the term “oxygenate,” refers to refers to oxygen-containing compounds having 1 to about 20 carbon atoms, 1 to about 10 carbon atoms, or 1 to about 4 carbon atoms. Exemplary oxygenates include alcohols, ethers, carbonyl compounds, e.g., aldehydes, ketones and carboxylic acids, and mixtures thereof. Particular non-limiting examples of oxygenates include methanol, ethanol, dimethyl ether, diethyl ether, methylethyl ether, di-isopropyl ether, dimethyl carbonate, dimethyl ketone, formaldehyde, acetaldehyde, acetic acid, and the like, and combinations thereof.

### II. Methods for Recovering Alkene from Process Gas Streams

Methods for recovering alkene from process gas streams are provided herein. The methods may comprise a condensation cycle including condensing the impurities and alkene gas in the process gas stream as well as a pressurization and/or a defrost cycle. Further, streams produced during the process may be advantageously recycled and utilized during the process and provide sufficient cooling of the process gas streams.

A. Condensation Cycle

As discussed above, a process gas stream may comprise various impurities in addition to alkene gas. Thus, it is necessary to efficiently and effectively remove these impurities from the process gas stream in order to sufficiently recover alkenes.

Therefore, in order to remove at least a portion of impurities present in a process gas stream, a condensation cycle is provided herein wherein a process gas stream may be cooled in a first condenser vessel with a first cooling medium under suitable conditions to produce a first condensate stream comprising at least a portion of the impurities and a first cooled process gas stream. The first condensate stream may be collected in a first condensate tank.

The process gas stream comprises alkene gas, e.g., C₂-C₁₀ alkenes, C₂-C₈ alkenes or C₂-C₄ alkenes. In particular the process gas stream comprises ethylene and/or propylene. Additionally, the process gas stream may comprise alkene gas (e.g., C₂-C₄ alkenes), in an amount, based on the total weight of the process gas stream, of about 1.0 wt %, at least about 10 wt %, at least about 15 wt %, at least about 20 wt %, at least about 25 wt %, at least about 30 wt %, at least about 35 wt %, at least about 40 wt %, at least about 45 wt %, at least about 50 wt %, at least about 55 wt %, at least about 60 wt %, at least about 65 wt %, at least about 70 wt %, at least about 75 wt %, at least about 80 wt %, at least about 85 wt %, at least about 90 wt %, at least about 95 wt %, at least about 99 wt % or about 99.5 wt %. It is understood herein that the amount of alkene gas provided herein corresponds to both a single alkene amount as well as combined amounts of alkenes, if one or more are present. For example, an alkene gas (e.g., C₂-C₄ alkenes) present in an amount of at least about 80 wt % encompasses a process stream comprising at least about 80 wt % ethylene as well as a process stream comprising at least about 80 wt % ethylene and propylene in combination. Additionally or alternatively, the process gas stream may comprise alkene gas (e.g., C₂-C₄ alkenes) in an amount, based on the total weight of the process gas stream, of about 1.0 wt % to about 99 wt %, about 1.0 wt % to about 90 wt %, about 1.0 wt % to about 80 wt %, about 1.0 wt % to about 60 wt %, about 1.0 wt % to about 40 wt %, about 1.0 wt % to about 20 wt %, about 1.0 wt % to about 20 wt %, about 20 wt % to about 99 wt %, about 40 wt % to about 99 wt %, about 60 wt % to about 99 wt %, about 70 wt % to about 99 wt %, about 80 wt % to about 99 wt %, about 80 wt % to about 95 wt %, about 80 wt % to about 90 wt %, or about 10 wt % to about 50 wt %.

A remaining portion, e.g., the balance, of the process gas stream may further comprise impurities, such as, but not limited to nitrogen, C₁-C₂₀ hydrocarbons (e.g., methane, ethane, propane, butane, butene, pentane, pentene, hexane, hexene, etc.), hydrogen, water and/or oxygenates (e.g., methyl acetate, ethyl acetate, vinyl acetate, methanol, dimethyl ether, acetaldehyde, etc.). In particular, the impurities are selected from the group consisting of nitrogen, methane, ethane, propane, butane, pentane, pentene, hexane, hexene, hydrogen, methyl acetate, ethyl acetate, vinyl acetate, methanol, dimethyl ether, acetaldehyde, water, and a combination thereof. For example, the impurities may be present in the process gas stream in an amount of at least about 1.0 wt %, at least about 5.0 wt %, at least about 10 wt %, at least about 15 wt %, at least bout 20 wt %, at least about 25 wt %, at least about 30 wt %, at least about 35 wt %, at least about 40 wt %, at least about 45 wt %, at least about 50 wt %, at least about 55 wt %, at least about 60 wt %, at least about 65 wt %, at least about 70 wt %, at least about 75 wt %, at least about 80 wt %, at least about 85 wt %, at least about 90 wt %, at least about 95 wt or about 99 wt %. Additionally or alternatively, the impurities may be present in the process gas stream in an amount of about 1.0 wt % to about 99 wt %, about 15 wt % to about 99 wt %, about 15 wt % to about 99 wt %, about 20 wt % to about 99 wt %, about 20 wt % to about 90 wt %, about 30 wt % to about 80 wt %, about 40 wt % to about 75 wt %, about 1.0 wt % to about 40 wt %, about 1.0 wt % to about 20, about 1.0 wt % to about 10 wt %, or about 1.0 wt % to about 5.0 wt. %. It is understood herein that the amount of impurities provided herein corresponds to both a single impurity amount as well as combined amounts of impurities, if one or more are present. For example, impurities present in an amount of at least about 10 wt % encompasses a process stream comprising at least about 10 wt % methanol as well as a process stream comprising at least about 10 wt % methanol and methane in combination.

In various aspects, the process gas stream may enter the first condenser vessel at any suitable temperature and/or pressure, for example, as determined by previous process steps and conditions for producing the process gas stream. For example, the process gas stream may enter the first condenser vessel at a temperature of about 50° C. or lower, about 40° C. or lower, about 30° C. or lower, about 20° C. or lower, about 10° C. or lower, about 0.0° C. or lower, about −10° C. or lower, about −20° C. or lower, about −30° C. or lower, about −40° C. or lower, about −50° C. or lower, about −60° C. or lower, about −70° C. or lower, about −80° C. or lower, about −90° C. or lower or about −100° C. Additionally or alternatively, the process gas stream may enter the first condenser vessel at a temperature of about −100° C. to about 50° C., about −100° C. to about 40° C., about −100° C. to about 20° C., about −100° C. to about 0.0° C., about −100° C. to about −10° C., about −100° C. to about −20° C., about −100° C. to about −30° C., about −100° C. to about −40° C., about −80° C. to about −0.0° C., about −80° C. to about −10° C., about −80° C. to about −20° C., about −80° C. to about −30° C., about −80° C. to about −40° C., about −50° C. to about 50° C., about −10° C. to about 50° C., or about 0.0° C. to about 40° C. Additionally, the process gas stream may enter the first condenser vessel at a pressure, optionally in combination with the above-described temperatures, of at least about 80 kPa, at least about 90 kPa, at least about 100 kPa, at least about 110 kPa, at least about 120 kPa, at least about 150 kPa, at least about 180 kPa, at least about 200 kPa, at least about 220 kPa, at least about 250 kPa, at least about 280 kPa. or about 300 kPa. For example, the process gas stream may enter the first condenser vessel at a pressure, optionally in combination with the above-described temperatures, of about 80 kPa to about 300 kPa, about 90 kPa to about 250 kPa, about 90 kPa to about 200 kPa, about 90 kPa to about 150 kPa or about 90 kPa to about 110 kPa. In particular, the process gas stream may enter the first condenser vessel at a temperature of about 40° C. or lower and a pressure of at least about 90 kPa.

In order to produce the first condensate stream, a first cooling medium may be circulated through the first condenser vessel at a temperature suitable for condensing at least a portion of the impurities present in the process gas stream to produce the first condensate stream comprising at least a portion of the impurities and the first cooled process gas stream comprising alkenes (e.g., C₂-C₄ alkenes), which may exit the first condenser vessel. Thus, the first cooled process gas stream may have impurities present in an amount less than an amount of impurities present in the process gas stream entering the first condenser vessel. Further, the first cooled process gas stream exiting the first condenser vessel may have a temperature of less than or equal to about 0.0° C., less than or equal to about −10° C., less than or equal to about −20° C., less than or equal to about −30° C., less than or equal to about −40° C., less than or equal to about −50° C., less than or equal to about −60° C., less than or equal to about −70° C., less than or equal to about −80° C., less than or equal to about −90° C., less than or equal to about −100° C., less than or equal to about −110° C., less than or equal to about −120° C., less than or equal to about −130° C., less than or equal to about −140° C., or about −150° C. In particular, the first cooled process gas stream may have a temperature of less than or equal to about −10° C. Additionally or alternatively, the first cooled process gas stream may have a temperature of about −150° C. to about 0.0° C., about −150° C. to about −10° C., about −140° C. to about −20° C., about −130° C. to about −30° C., about −120° C. to about −40° C. or about −110° C. to about −50° C.

In order to recover the alkenes (e.g., C₂-C₄ alkene) from the process gas stream, the process cycle may further comprise cooling the first cooled process gas stream comprising alkenes (e.g., C₂-C₄ alkene) in a second condenser vessel under suitable conditions to produce a second condensate. A second cooling medium may be circulated through the second condenser vessel at a temperature suitable for condensing at least a portion of the alkene gas (e.g., C₂-C₄ alkenes) present in the process gas stream to produce the second condensate comprising alkenes (e.g., C₂-C₄ alkenes). The second condensate comprising alkenes (e.g., C₂-C₄ alkenes) may be collected in a second condensate tank. Additionally or alternatively, the second condensate exiting the second condenser vessel may have a temperature of about −170° C. to about −104° C., about −170° C. to about −120° C., or about −170° C. to about −140° C., or about −150° C. to about −104° C.

In various aspects, the second condensate may comprise at least about 40 wt %, at least about 50 wt %, at least about 60 wt %, at least about 70 wt %, at least about 80 wt %, at least about 90 wt %, at least about 95 wt %, or about 99 wt % of the alkenes (e.g., C₂-C₄ alkenes), which were present in the process gas stream. For example, the second compensate may comprise about 40 wt % to about 99 wt %, about 50 wt % to about 99 wt %, about 70 wt % to about 99 wt %, about 80 wt % to about 99 wt %, or about 90 wt % to about 99 wt % of the alkenes (e.g., C₂-C₄ alkenes), which were present in the process gas stream, In particular, at least about 80 wt % of the alkenes (e.g., C₂-C₄ alkenes) present in the process gas stream may be present in the second condensate.

Examples of suitable first and second cooling mediums include, but are not limited to liquid nitrogen and/or gaseous nitrogen. The first and/or second cooling medium (e.g., liquid and/or gaseous nitrogen) may be provided and/or circulated in the first and/or second condenser vessel at a temperature of at least about −210° C., at least about −196° C., at least about −190° C., at least about −180° C., at least about −170° C., at least about −160° C., or at least about −150° C. For example, the first and/or second cooling medium (e.g., liquid and/or gaseous nitrogen) may be provided and/or circulated in the first and/or second condenser vessel at a temperature of about −210° C. to about −150° C., about −196° C. to about −150° C., about −190° C. to about −150° C., or about −180° C. to about −160° C. Additionally, the first and/or second cooling medium (e.g., liquid nitrogen and/or gaseous nitrogen) may be provided and/or circulated in the first and/or second condenser vessel, optionally in combination with the above-described temperatures, at a pressure of less than or equal to about 1600 kPa, less than or equal to about 1500 kPa, less than or equal to about 1300 kPa, less than or equal to about 1200 kPa, less than or equal to about 1000 kPa, less than or equal to about 800 kPa, less than or equal to about 700 kPa, less than or equal to about 500 kPa, less than or equal to about 300 kPa, or about 100 kPa. For example, the first and/or second cooling medium (e.g., liquid and/or gaseous nitrogen) may be provided and/or circulated in the first and/or second condenser vessel, optionally in combination with the above-described temperatures, at a pressure of about 100 kPa to about 1600 kPa, about 300 kPa to about 1500 kPa, about 500 kPa to about 1500 kPa, about 700 kPa to about 1300 kPa or about 800 kPa to about 1200 kPa. In particular, the first and/or second cooling medium (e.g., liquid and/or gaseous nitrogen) may be provided and/or circulated in the first and/or second condenser vessel at a temperature of at least about −196° C. and/or at a pressure of less than or equal to about 1500 kPa (e.g., about −170° C. and about 1000 kPa). Additionally or alternatively, the first condensate collecting in the at least one condenser vessel may have a temperature of about −170° C. to about −104° C., about −170° C. to about −120° C., or about −170° C. to about −140° C., or about −150° C. to about −104° C. The first and/or second condenser vessel may each independently comprise heat exchangers, such as a coil heat exchanger, a shell and tube heat exchanger and a plate heat exchanger.

During the condensation cycle, a first vent gas may also be produced in the second condenser vessel. The vent gas may comprise the non-condensable components of the process gas stream. For example, the first vent gas may primarily comprise (e.g., ≥about 90 wt %, ≥about 95 wt %, ≥about 98 wt %, ≥about 99 wt %, or about 99.5 wt %) the impurities as described herein present in the process gas stream, such as but not limited to nitrogen, hydrogen and/or methane. In particular, the first vent gas comprises nitrogen. Additionally or alternatively, the first vent gas may comprise trace amounts (e.g., ≤about 5.0 wt %, ≤about 2.0 wt %) of alkenes (e.g., C₂-C₄ alkenes) and/or alkanes (e.g., methane, ethane, etc.). Further, during the process, the first vent gas may be continuously removed from the second condenser vessel or when the pressure drop in the second condenser vessel reaches a predetermined value. For example, where the second condenser vessel comprises a coil heat exchanger, the first vent gas may be removed from the second condenser vessel when the pressure in the second condenser vessel reaches greater than about 100 kPa, at least about 200 kPa, at least about 300 kPa, at least about 400 kPa or about 500 kPa. Additionally or alternatively, the first vent gas may be removed from the second condenser vessel when the pressure in the condenser vessel is about 100 kPa to about 500 kPa, about 200 kPa to about 400 kPa, or about 300 kPa to about 500 kPa. Further, in some aspects, the first vent gas may exit the second condenser vessel at a temperature of about −170° C. to about −104° C., or about −160° C. to about −104° C. and/or at pressure of at least about 100 kPa, at least about 200 kPa, at least about 300 kPa, at least about 400 kPa, or at least about 500 kPa.

Optionally, the condensation cycle may further comprising cooling the process gas stream in a pre-cooling apparatus prior to the process gas entering the first condenser vessel. Suitable pre-cooling apparatuses include, but are not limited to a mechanical chiller, such as a water chiller or ethylene glycol chiller, and a heat exchanger. In the pre-cooling apparatus, the process gas stream may be cooled to a temperature of about −40° C. or higher, −30° C. or higher, −10° C. or higher, 0.0° C. or higher, 10° C. or higher, 20° C. or higher, 30° C. or higher, 40° C. or higher, or about 50° C. In particular, the process gas stream in the pre-cooling apparatus may be cooled to a temperature of about −40° C. or higher. Additionally or alternatively, in the pre-cooling apparatus, the process gas stream may be cooled to a temperature of about −40° C. to about 50° C., about −20° C. to about 50° C., or about 0.0° C. to about 50° C.

In various aspects, the condensation cycle may run for about 1.0 hour to about 72 hours, about 3.0 hours to about 48 hours, about 6.0 hours to about 24 hours or about 6.0 hours to about 18 hours. In particular, the condensation cycle may run for about 6 hours to about 24 hours or about 8 hours to about 12 hours.

B. Pressurization Cycle

The process for recovering alkenes (e.g., C₂-C₄ alkenes) may further comprise a pressurization cycle. The pressurization cycle may comprise halting flow of the process gas stream and the first cooling medium to the first condenser vessel, flow of the first condensate to the first condensate tank, flow of the first cooled process gas stream and the second cooling medium to the second condenser vessel and/or flow of the second condensate to the second condensate tank. Optionally, the process gas stream may be directed to another condenser vessel in series where it may undergo a condensation cycle as described herein.

Additionally, the pressurization cycle may comprise heating the second condensate tank to produce pressurized liquid alkene (e.g., C₂-C₄ alkene) and an alkene (e.g., C₂-C₄ alkene) vent gas. The heat provided to the second condensate tank may vaporize at least a portion of the alkenes (e.g., C₂-C₄ alkene) in the first condensate to produce pressurized liquid alkene (e.g., C₂-C₄ alkene) and an alkene (e.g., C₂-C₄ alkene) vent gas. The alkene (e.g., C₂-C₄ alkene) vent gas may also comprise other components, such as methane and/or ethane. Any suitable means for providing heat to the second condensate tank may be used, for example, heat may be provided via a heater (e.g., electric heater), via heated gaseous nitrogen, via heated air or via an ambient vaporizer. Further, the heat may be provided at suitable temperature for a suitable amount of time to produce pressurized liquid alkene (e.g., C₂-C₄ alkene) at a desirable pressure as determined by the needs of the process, for example, at a pressure of about 100 kPa to about 1500 kPa, about 200 kPa to about 800 kPa or about 300 kPa to about 500 kPa. For example, the pressurized liquid alkene (e.g., C₂-C₄ alkene) may be maintained at a temperature of at least about 100 kPa. Additionally or alternatively, the pressurized liquid alkene (e.g., C₂-C₄ alkene) may have a temperature of less than about −104° C., e.g., about −170° C. to about −104° C., about −170° C. to about −120° C., about −170° C. to about −140° C., or about −150° C. to about −104° C.

It is contemplated herein, that multiple condensation cycles (e.g., a first condensation, a second condensation cycle, etc.), pressurization cycles (e.g., a first pressurization cycle, a second pressurization cycle, etc.), and defrost cycles (described below) (e.g., a first defrost cycle, a second defrost cycle, etc.) may be performed in corresponding condenser vessels having the same steps as the condensation cycle described herein, the pressurization cycle described herein, and the defrost cycle as described below. For example, the condensation cycle described herein may correspond to a first condensation cycle and the pressurization cycle described herein may correspond to a first pressurization cycle. In such instance, the first pressurization cycle may further comprise optionally recycling at least a portion of the pressurized liquid alkene (e.g., C₂-C₄ alkene) to a second condensation cycle comprising the same steps as the first condensation cycle for use as the first cooling medium in the second condensation cycle. Thus, the pressurized liquid alkene (e.g., C₂-C₄ alkene) may advantageously provide cooling instead of or in addition to liquid and/or gaseous nitrogen in another condenser vessel undergoing a condensation cycle. For example, during the second condensation cycle, the process gas stream comprising alkene (e.g., C₂-C₄ alkene) and impurities may be cooled in a third condenser vessel (similar to the first condenser vessel) with the first cooling medium under suitable conditions to produce a third condensate (similar to the first condensate) and a second cooled process gas stream (similar to the first cooled process gas stream). Advantageously, the first cooling medium used for cooling in the third condenser vessel may be the pressurized liquid alkene (e.g., C₂-C₄ alkene) produced during the first condensation cycle and first pressurization cycle, optionally supplemented with an additional suitable cooling medium. The third condensate may comprise at least a portion of the impurities and the second cooled process gas stream may comprise C₂-C₄ alkene having a temperature of less than or equal to −10° C. The third condensate may be collected in a third condensate tank (similar to the first condensate tank). Additionally, the second cooled process gas stream comprising alkene (e.g., C₂-C₄ alkene) may be further cooled in a fourth condenser vessel (similar to the second condenser vessel) with the second cooling medium under suitable conditions to produce a fourth condensate stream (similar to the second condensate stream) comprising alkene (e.g., C₂-C₄ alkene) and a second vent gas stream (similar to the first vent gas stream). The fourth condensate stream may be collected in a fourth condensate tank (similar to the second condensate tank) and/or in the second condensate tank. It is further contemplated herein, that the condenser vessels used during the second condensation cycle may undergo a second pressurization cycle as described above. Optionally, pressurized liquid alkene (e.g., C₂-C₄ alkene) produced during the second pressurization cycle may be recycled for use as at least a portion of the first cooling medium in the first condenser vessel during the first condensation cycle.

In various aspects, the pressurization cycle may run for about 1.0 hour to about 72 hours, about 3.0 hours to about 48 hours, about 6.0 hours to about 24 hours or about 6.0 hours to about 18 hours. In particular, the pressurization cycle may run for about 6 hours to about 24 hours or about 8 hours to about 12 hours.

C. Defrost Cycle

The methods described herein may further comprise a defrost cycle to melt any frozen alkenes and/or impurities in the condenser vessels (e.g., first condenser vessel, second condenser vessel). The defrost cycle may be performed as needed by the process. For example, the defrost cycle may be commenced when the pressure drop within the condenser vessels (e.g., first condenser vessel, second condenser vessel) increases to an undesirable level and/or when the level of condensate in the condensate tanks (e.g., first condensate tank, second condensate tank) is too high. The defrost cycle may comprise halting the flow of the process gas stream and the first cooling medium to the first condenser vessel, flow of the first condensate to the first condensate tank, flow of the first cooled process gas stream and the second cooling medium to the second condenser vessel and/or flow of the second condensate to the second condensate tank. Then the condenser vessels (e.g., first condenser vessel, second condenser vessel) may be heated to produce at least a fifth condensate stream comprising alkenes (e.g., C₂-C₄ alkene). The heating of the condenser vessels (e.g., first condenser vessel, second condenser vessel) may be provided by any suitable means for defrosting the condenser vessel. For example, heating may be provided by gaseous nitrogen. The gaseous nitrogen may be heated to a suitable temperature (e.g., greater than about −104° C. up to about 25° C.) prior to introduction into the condenser vessels (e.g., first condenser vessel, second condenser vessel). The defrost cycle may further comprise draining the fifth condensate stream from the second condenser vessel to collect in the second condensate tank or a fifth condensate tank. It is further contemplated herein, that the condenser vessels used during the second condensation cycle (e.g., third condenser vessel, fourth condenser vessel) may undergo a defrost cycle as described above.

In various aspects, the defrost cycle may run for about 1.0 hour to about 18 hours, about 1.0 hour to about 12 hours, about 1.0 hour to about 6.0 hours, about 1.0 hour to about 3.0 hours, or about 1.0 hour to about 2.0 hours. In particular, the defrost cycle may run for about 1.0 hour to about 3.0 hours.

Additionally, the first condensation cycle, the second condensation cycle, the first pressurization cycle, the second pressurization cycle, and the defrost cycle(s) may run in parallel in two or more condenser vessels and associated condensate tanks, which may be in series. For example, it is contemplated herein that while the first condenser vessel, the first condensate tank, the second condenser vessel, and the second condensate tank are undergoing a first condensation cycle, the third condensate tank, and the fourth condensate tank may be undergoing a second pressurization cycle. Alternatively, it is contemplated herein that while the first condensate tank, and the second condensate tank are undergoing a first pressurization cycle, the third condenser vessel, the third condensate tank, the fourth condenser vessel, and the fourth condensate tank are undergoing a second condensation cycle.

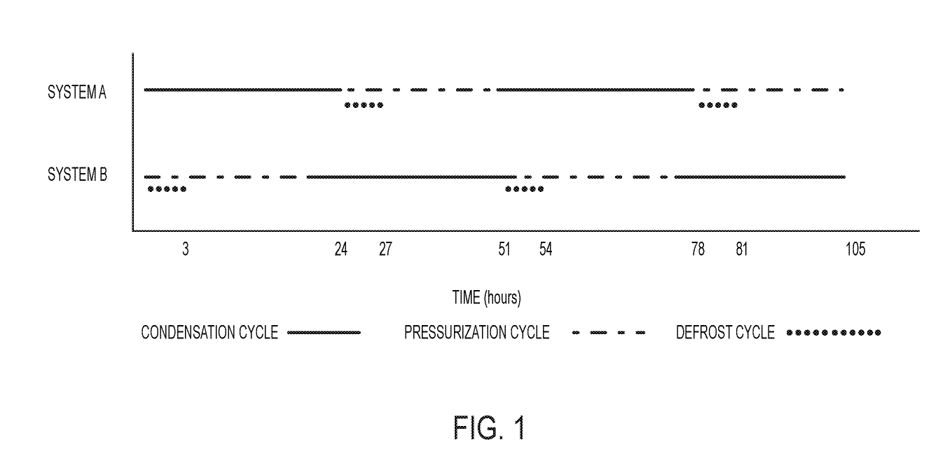

Further, it is contemplated herein that a sequence of respective cycles in two systems (System A and System B) running in parallel may be as illustrated in FIG. 1. For example, System A, which comprises two or more condenser vessels and associated condensate tanks, may be undergoing a condensation cycle followed by a defrost cycle and a pressurization cycle while System B, which comprises two or more condenser vessels and associated condensate tanks, may be undergoing a defrost cycle and a pressurization cycle followed by a condensation cycle and so on. As shown in FIG. 1, at least a portion of a defrost cycle and at least a portion of a pressurization cycle may optionally run simultaneously. For example, in System A, while the condenser vessel(s) may be undergoing a defrost cycle, the associated condensate tank(s) may undergo a pressurization cycle.

### III. Systems for Recovering Alkenes from Process Gas Streams

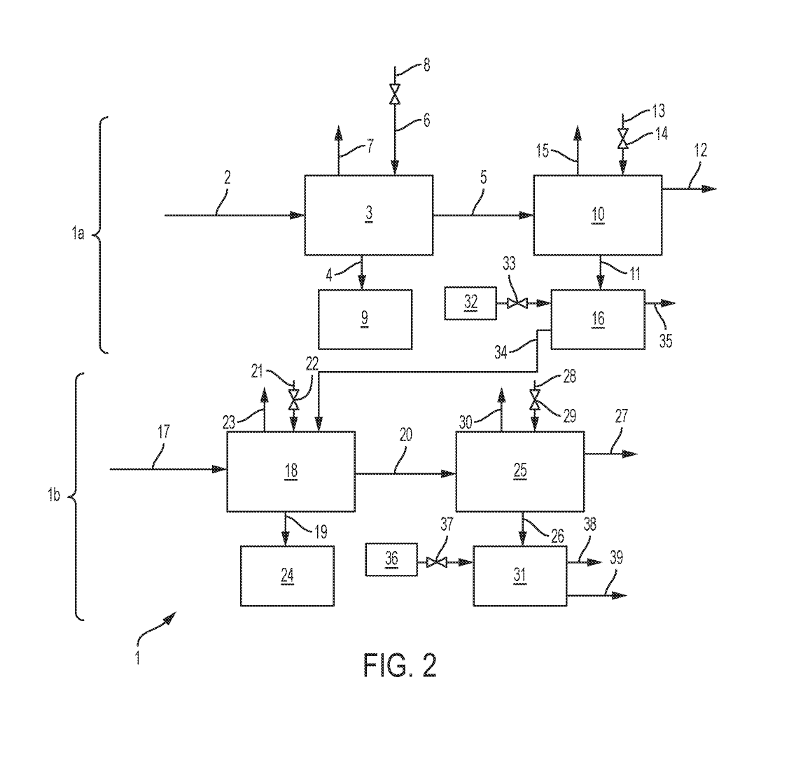

Systems for recovering alkene (e.g., C₂-C₄ alkene) from a process gas stream as described herein are also provided. Referring to FIG. 2, the system 1 may comprise a first sub-system 1a of condenser vessels and condensate tanks and a second sub-system 1b of condenser vessels and condensate tanks. The first sub-system 1a may comprise a first process gas stream 2 comprising alkenes (e.g., C₂-C₄ alkene) and impurities, which is provided to a first condenser vessel 3 via a first inlet (not shown), for example, during a condensation cycle (e.g., first condensation cycle) as described herein, wherein a first condensate stream 4 and a first cooled process gas stream 5 are produced. In particular, the first process gas stream 2 may comprise alkenes (e.g., C₂-C₄ alkene) as described herein and impurities (e.g., nitrogen, hydrogen, water, C₁-C₁₀ hydrocarbons, oxygenates) as described herein. In certain aspects, the alkenes are ethylene and/or propylene.

Additionally, in order to produce the first condensate stream 4 comprising at least a portion of the impurities as described herein and the first cooled process gas stream 5 as described herein (e.g., having a temperature of less than or equal to −10° C.), the system 1 may further comprise a first cooling medium stream 6 provided via a second inlet (not shown), as controlled by a valve 8, which may be circulated through the first condenser vessel 3, at a temperature suitable for condensing at least a portion of the impurities present in the process gas stream 2 to produce the first condensate stream 4 comprising impurities. The first condenser vessel 3 may comprise, consist essentially of, or consist of a first heat exchanger (e.g., shell and tube heat exchanger, plate heat exchanger, coil heat exchanger). The first cooling medium stream 6 may comprise a suitable cooling medium as described herein, for example, liquid nitrogen and/or gaseous nitrogen. As the first cooling medium stream 6 (e.g., gaseous and/or liquid nitrogen) circulates through the first condenser vessel 3, it may be heated and exit the system 1 as a first spent cooling medium stream 7 via a first outlet (not shown). Additionally or alternatively, a valve (not shown) may be present on the first spent cooling medium stream 7 for controlling the cooling medium circulating through the first condenser vessel 3. In various aspects, at least a portion of the first spent cooling medium stream 7 may comprise gaseous nitrogen.

A first condensate tank 9 may be present in the first sub-system 1a for collection of the first condensate stream 4, which may be removed via a third outlet (not shown) in the first condenser vessel 3, for example, as the first condensate stream 4 drains from the first condenser vessel 3 during a condensation cycle (e.g., first condensation cycle, second condensation cycle) as described herein. The first condensate tank 9 may comprise a third inlet (not shown) for providing the first condensate stream 4.

The first sub-system 1a may further comprise a second condenser vessel 10 for alkene (e.g., C₂-C₄ alkene) recovery. In particular, the first cooled process gas stream 5 may exit the first condenser vessel 3 via a second outlet (not shown) and enter the second condenser vessel 10 via a fourth inlet (not shown). The second condenser vessel 10 may be operated under suitable conditions to produce a second condensate stream 11 comprising alkenes (e.g., C₂-C₄ alkene) and a first vent gas stream 12 as described herein. The first vent gas stream 12 may exit the second condenser vessel 10 via a fifth outlet (not shown) and may primarily comprise (e.g., ≥about 90 wt %, ≥about 95 wt %, ≥ about 98 wt %, %, ≥ about 99 wt %, or about 99.5 wt %) the non-condensable components of the first process gas stream 2, e.g., hydrogen, nitrogen, ethane and/or methane. In particular, the first vent gas stream 12 comprises nitrogen.

In order to produce the second condensate stream 11 as described herein and the first vent gas stream 12 as described herein, the first sub-system 1a may further comprise a second cooling medium stream 13 provided via a fifth inlet (not shown), as controlled by valve 14, which may be circulated through the second condenser vessel 10, at a temperature suitable for condensing at least a portion of the alkenes (e.g., C₂-C₄ alkene) present in the first process gas stream 2 to produce the second condensate stream 11 comprising alkenes (e.g., C₂-C₄ alkene). The second condenser vessel 10 may comprise, consist essentially of, or consist of a second heat exchanger (e.g., shell and tube heat exchanger, plate heat exchanger, coil heat exchanger). The second cooling medium stream 13 may comprise a suitable cooling medium as described herein, for example, liquid nitrogen and/or gaseous nitrogen. As the second cooling medium stream 13 (e.g., gaseous and/or liquid nitrogen) circulates through the second condenser vessel 10, it may be heated and exit the system 1 as a second spent cooling medium stream 15 via a fourth outlet (not shown). Additionally or alternatively, a valve (not shown) may be present on the second spent cooling medium stream 15 for controlling the cooling medium circulating through the second condenser vessel 10. In various aspects, at least a portion of the second spent cooling medium stream 15 may comprise gaseous nitrogen.

A second condensate tank 16 may be present in the system 1 for collection of the second condensate stream 11, which may be removed via a sixth outlet (not shown) in the second condenser vessel 10, for example, as the second condensate stream 11 drains from the second condenser vessel 10 during a condensation cycle (e.g., first condensation cycle) as described herein. The second condensate tank 16 may comprise a sixth inlet (not shown) for providing the second condensate stream 11.

The first sub-system 1a may further comprise a first means for providing heat 32 and valve 33 for providing heat to the second condensate tank 16 to produce a first pressurized liquid alkene (e.g., C₂-C₄ alkene) stream 34 and a first alkene (e.g., C₂-C₄ alkene) vent gas stream 35, for example, during a pressurization cycle as described herein (e.g., a first pressurization cycle). The first means for providing heat 32 may be a heater as described herein, an ambient vaporizer, heated air, or heated gaseous nitrogen. The second condensate tank 16 may further comprise a seventh outlet (not shown) for removal of the first pressurized liquid alkene (e.g., C₂-C₄ alkene) stream 34 and an eighth outlet (not shown) for removal of the first alkene (e.g., C₂-C₄ alkene) vent gas stream 35.

Advantageously, while the first process gas stream 2 in the first sub-sytem 1a undergoes a condensation cycle as described herein (e.g., a first condensation cycle), a pressurization cycle as described herein (e.g., a first pressurization cycle) and a defrost cycle as described herein, one or more condensation cycles (e.g., a second condensation cycle), pressurization cycles (e.g., a second pressurization cycle) and/or defrost cycles may be running in additional condenser vessels. For example, system 1 may further comprise a second sub-system 1b comprsing a second process gas stream 17, which is provided to a third condenser vessel 18 via a seventh inlet (not shown), for example, during a second condensation cycle as described herein, wherein a third condensate stream 19 and a second cooled process gas stream 20 are produced. In particular, the second process gas stream 17 may comprise alkenes (e.g., C₂-C₄ alkene) as described herein and impurities (e.g., nitrogen, hydrogen, water, C₁-C₁₀ hydrocarbons, oxygenates) as described herein. The first process gas stream 2 and the second process gas stream 17 may be from the same or different source.

In order to produce the third condensate stream 19 comprising at least a portion of the impurities as described herein and the second cooled process gas stream 20 as described herein (e.g., having a temperature of less than or equal to −10° C.), the system 1 may further comprise a third cooling medium stream 21 provided via an eighth inlet (not shown), as controlled by a valve 22, which may be circulated through the third condenser vessel 18, at a temperature suitable for condensing at least a portion of the impurities present in the second process gas stream 17 to produce the second condensate stream 19 comprising impurities. The third condenser vessel 18 may comprise, consist essentially of, or consist of a third heat exchanger (e.g., shell and tube heat exchanger, plate heat exchanger, coil heat exchanger). The third cooling medium stream 21 may comprise a suitable cooling medium as described herein, for example, liquid nitrogen and/or gaseous nitrogen. As the third cooling medium stream 21 (e.g., gaseous and/or liquid nitrogen) circulates through the third condenser vessel 18, it may be heated and exit the system 1 as a third spent cooling medium stream 23 via a ninth outlet (not shown). Additionally or alternatively, a valve (not shown) may be present on the third spent cooling medium stream 23 for controlling the cooling medium circulating through the third condenser vessel 18. In various aspects, at least a portion of the third spent cooling medium stream 23 may comprise gaseous nitrogen.

Advantageously, at least a portion of the first pressurized liquid alkene (e.g., C₂-C₄ alkene) stream 34 may be recycled for use as the cooling medium in the third condenser vessel 18. In such instances, it is contemplated herein that the first sub-system 1a may be undergoing a pressurization cycle (e.g., first pressurization cycle) while the second sub-system 1b may be undergoing a condensation cycle (e.g., second condensation cycle).

In various aspects, only the first pressurized liquid alkene (e.g., C₂-C₄ alkene) stream 34 may be provided to the third condenser vessel 18 for cooling the second process gas stream 17 and may be considered the third cooling medium stream. In such instances, the third cooling medium stream 21 comprising liquid and/or gaseous nitrogen may not be needed. In other embodiments, the third cooling medium stream 21 may supplement the first pressurized liquid alkene (e.g., C₂-C₄ alkene) stream 34 to sufficiently cool the second process gas stream 17.

A third condensate tank 24 may be present in the second subsystem 1b for collection of the third condensate stream 19, which may be removed via an eleventh outlet (not shown) in the third condenser vessel 18, for example, as the third condensate stream 19 drains from the third condenser vessel 18 during a condensation cycle (e.g., first condensation cycle, second condensation cycle) as described herein. The third condensate tank 24 may comprise a ninth inlet (not shown) for providing the third condensate stream 19. It is contemplated herein, that the first condensate stream 4 and the third condensate stream 19 may be collected in the same condensate tank.

The second sub-system 1b may further comprise a fourth condenser vessel 25 for alkene (e.g., C₂-C₄ alkene) recovery via a fourth condensate stream 26. In particular, the second cooled process gas stream 20 may exit the third condenser vessel 18 via an tenth outlet (not shown) and enter the fourth condenser vessel 25 via a tenth inlet (not shown). The fourth condenser vessel 25 may be operated under suitable conditions to produce a fourth condensate stream 26 comprising alkenes (e.g., C₂-C₄ alkene) and a second vent gas stream 27 as described herein. The second vent gas stream 27 may exit the fourth condenser vessel 25 via an thirteenth outlet (not shown) and may primarily comprise (e.g., ≥about 90 wt %, ≥about 95 wt %, ≥ about 98 wt %, %, ≥about 99 wt % %, or about 99.5 wt %) the non-condensable components of the second process gas stream 17, e.g., hydrogen, nitrogen, ethane and/or methane. In particular, the second vent gas stream 27 comprises nitrogen.

In order to produce the fourth condensate stream 26 as described herein and the second vent gas stream 27 as described herein, the second sub-system 1b may further comprise a fourth cooling medium stream 28 provided via an eleventh inlet (not shown), as controlled by valve 29, which may be circulated through the fourth condenser vessel 21, at a temperature suitable for condensing at least a portion of the alkenes (e.g., C₂-C₄ alkene) present in the second process gas stream 17 to produce the fourth condensate stream 26 comprising alkenes (e.g., C₂-C₄ alkene). The fourth condenser vessel 25 may comprise, consist essentially of, or consist of a fourth heat exchanger (e.g., shell and tube heat exchanger, plate heat exchanger, coil heat exchanger). The fourth cooling medium stream 28 may comprise a suitable cooling medium as described herein, for example, liquid nitrogen and/or gaseous nitrogen. As the fourth cooling medium stream 28 (e.g., gaseous and/or liquid nitrogen) circulates through the fourth condenser vessel 25, it may be heated and exit the system 1 as a fourth spent cooling medium stream 30 via a twelfth outlet (not shown). Additionally or alternatively, a valve (not shown) may be present on the fourth spent cooling medium stream 30 for controlling the cooling medium circulating through the fourth condenser vessel 25. In various aspects, at least a portion of the fourth spent cooling medium stream 30 may comprise gaseous nitrogen.

A fourth condensate tank 31 may be present in the second subsystem 1b for collection of the fourth condensate stream 26, which may be removed via a fourteenth outlet (not shown) in the fourth condenser vessel 25, for example, as the fourth condensate stream 26 drains from the fourth condenser vessel 25 during a condensation cycle (e.g., first condensation cycle, second condensation cycle) as described herein. The fourth condensate tank 31 may comprise a twelfth inlet (not shown) for providing the fourth condensate stream 26.

The second sub-system 1b may further comprise a second means for providing heat 36 and valve 37 for providing heat to the fourth condensate tank 31 to produce a second pressurized liquid alkene (e.g., C₂-C₄ alkene) stream 39 and a second alkene (e.g., C₂-C₄ alkene) vent gas stream 38, for example, during a second pressurization cycle as described herein. The second means for providing heat 36 may be a heater as described herein, an ambient vaporizer, heated air, or heated gaseous nitrogen. The fourth condensate tank 31 may further comprise a fifteenth outlet (not shown) for removal of the second pressurized liquid alkene (e.g., C₂-C₄ alkene) stream 39 and a sixteenth outlet (not shown) for removal of the second alkene (e.g., C₂-C₄ alkene) vent gas stream 38. Although not shown, it is contemplated herein, that at least a portion of the second pressurized liquid alkene (e.g., C₂-C₄ alkene) stream 39 advantageously may be recycled for use as the cooling medium stream in the first condenser vessel 3, optionally supplemented with an additional suitable cooling medium. In such instances, it is contemplated herein that the first sub-system 1a may be undergoing a condensation cycle (e.g., first condensation cycle) while the second sub-system 1b may be undergoing a pressurization cycle (e.g., second pressurization cycle). In various aspects, it is also contemplated herein that the first sub-system 1a and the second sub-system 1b may be undergoing a sequence of cycles as shown in FIG. 1, where the first sub-system 1a can be considered to be “System A” and the second sub-system 1b can be considered to be “System B.”

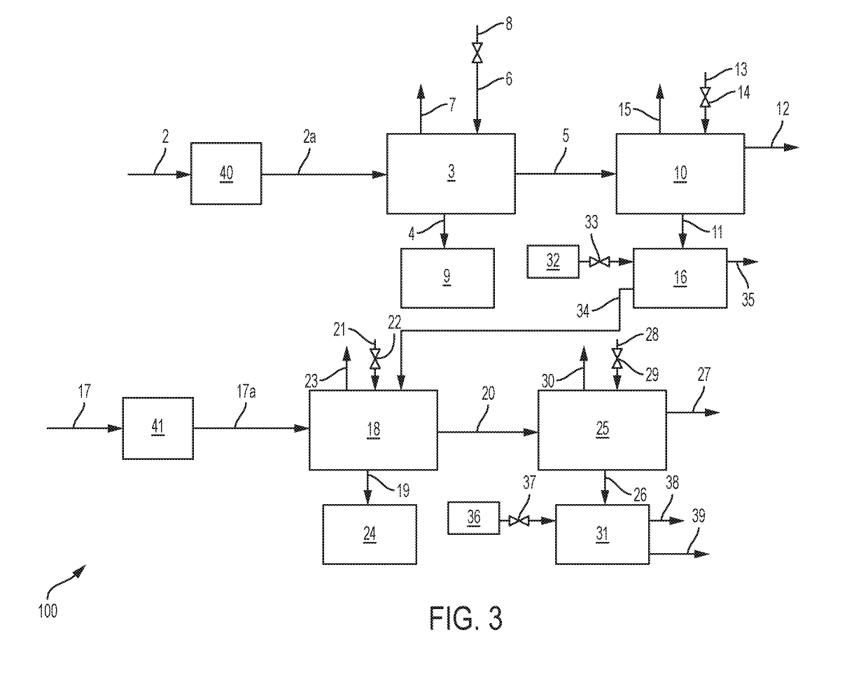

In an alternative embodiment, as shown in FIG. 3, a system 100 for recovering alkene (e.g., C₂-C₄ alkene) from a process gas stream may further comprise a pre-cooling apparatus 40 and/or 41 for cooling the first process gas stream 2 and/or the second process gas stream 17 to produce a pre-cooled first process gas stream 2a and/or a pre-cooled second process gas stream 17a. The pre-cooled first process gas stream 2a and/or the pre-cooled second process gas stream 17a then may be introduced into the first condenser vessel 3 and/or the third condenser vessel 18, respectively. Suitable pre-cooling apparatuses include, but are not limited to a mechanical chiller, such as a water chiller or ethylene glycol chiller, and a heat exchanger. In the pre-cooling apparatus, the first process gas stream 2 and/or the second process gas stream 17 may be cooled to a temperature as described herein, e.g., about −40° C. or higher, −30° C. or higher, −10° C. or higher, 0.0° C. or higher, 10° C. or higher, 20° C. or higher, 30° C. or higher, 40° C. or higher, or about 50° C.

### IV. Further Embodiments

The invention can additionally or alternatively include one or more of the following embodiments.

### Embodiment 1

A method for recovering C₂-C₄ alkene (e.g., ethylene and/or propylene) from a process gas stream comprising: a first condensation cycle comprising: cooling the process gas stream comprising C₂-C₄ alkene (e.g., ethylene and/or propylene) and impurities (e.g., nitrogen, hydrogen, water, C₁-C₂₀ hydrocarbons, and/or oxygenates) in a first condenser vessel with a first cooling medium (e.g., liquid and/or gaseous nitrogen) under suitable conditions to produce a first condensate comprising at least a portion of the impurities (e.g., nitrogen, hydrogen, water, C₁-C₂₀ hydrocarbons, and/or oxygenates) and a first cooled process gas stream comprising C₂-C₄ alkene (e.g., ethylene and/or propylene) having a temperature of less than or equal to −10° C.; collecting the first condensate in a first condensate tank; cooling the first cooled process gas stream comprising C₂-C₄ alkene (e.g., ethylene and/or propylene) in a second condenser vessel with a second cooling medium (e.g., liquid and/or gaseous nitrogen) under suitable conditions to produce a second condensate comprising C₂-C₄ alkene (e.g., ethylene and/or propylene) and a first vent gas stream; and collecting the second condensate in a second condensate tank; and a pressurization cycle comprising: heating the second condensate tank to produce pressurized liquid C₂-C₄ alkene (e.g., ethylene and/or propylene); and optionally, recycling at least a portion of the pressurized liquid C₂-C₄ alkene (e.g., ethylene and/or propylene) to a second condensation cycle comprising the same steps as the first condensation cycle for use as the first cooling medium.

### Embodiment 2

The method of embodiment 1, wherein the process gas stream enters the first condenser vessel at a temperature of about 40° C. or lower and a pressure of at least about 90 kPa.

### Embodiment 3

The method of embodiment 1 or 2, wherein the first vent gas stream comprises nitrogen.

### Embodiment 4

The method of any one of the previous embodiments, wherein the first condenser vessel and/or the second condenser vessel each independently comprises a coil heat exchanger, a shell and tube heat exchanger or a plate heat exchanger.

### Embodiment 5

The method of any one of the previous embodiments, wherein the second cooling medium is provided at a temperature of at least about −196° C. and a pressure of less than or equal to about 1500 kPa.

### Embodiment 6

The method of any one of the previous embodiments, wherein the pressurized liquid C₂-C₄ alkene is maintained at a pressure of at least about 100 kPa.

### Embodiment 7

The method of any one of the previous embodiments, wherein at least about 80 wt % of the C₂-C₄ alkenes present in the process gas stream are present in the second condensate.

### Embodiment 8

The method of any one of the previous embodiments, wherein the condensation cycle runs for about 6.0 hours to about 24 hours and the pressurization cycle runs for about 6.0 to about 24 hours.

### Embodiment 9

The method of any one of the previous embodiments further comprises cooling the process gas stream in a pre-cooling apparatus prior to introduction into the first condenser vessel, wherein the process gas stream is cooled to a temperature of about −40° C. or higher.

### Embodiment 10

A system for recovering C₂-C₄ alkene (e.g., ethylene and/or propylene) from a process gas stream comprising: a first process gas stream comprising C₂-C₄ alkene (e.g., ethylene and/or propylene); a first cooling medium stream a first condensate stream comprising at least a portion of the impurities; a first cooled process gas stream having a temperature of less than or equal to −10° C.; a first vent gas stream; a second cooling medium stream; a second condensate stream comprising C₂-C₄ alkene (e.g., ethylene and/or propylene); a second process gas stream comprising C₂-C₄ alkene (e.g., ethylene and/or propylene); a third cooling medium stream; a third condensate stream comprising at least a portion of the impurities; a second cooled process gas stream having a temperature of less than or equal to −10° C.; a second vent gas stream; a fourth cooling medium stream; a fourth condensate stream comprising C₂-C₄ alkene (e.g., ethylene and/or propylene); a first pressurized liquid C₂-C₄ alkene (e.g., ethylene and/or propylene) stream; a first C₂-C₄ alkene (e.g., ethylene and/or propylene) vent gas stream; a first condenser vessel operated under suitable conditions to produce the first condensate stream comprising at least a portion of the impurities and the first cooled process gas stream, wherein the first condenser vessel comprises: a first heat exchanger; a first inlet for providing the first process gas stream; a second inlet for providing the first cooling medium; a first outlet for removal of a first spent cooling medium; a second outlet for removal of the first cooled process gas stream; and a third outlet for removal of the first condensate stream; a first condensate tank comprising: a third inlet for providing the first condensate stream; a second condenser vessel operated under suitable conditions to produce the second condensate stream comprising C₂-C₄ alkene (e.g., ethylene and/or propylene) and the first vent gas stream, wherein the second condenser vessel comprises: a second heat exchanger; a fourth inlet for providing the first cooled process gas stream; a fifth inlet for providing the second cooling medium; a fourth outlet for removal of a second spent cooling medium; a fifth outlet for removal of the first vent gas stream; and a sixth outlet for removal of the second condensate stream; a second condensate tank comprising: a sixth inlet for providing the second condensate stream; a seventh outlet for removal of the first pressurized liquid C₂-C₄ alkene stream; and an eighth outlet for removal the first C₂-C₄ alkene vent gas stream; a first means for providing heat to the second condensate tank; a third condenser vessel operated under suitable conditions to produce the third condensate stream comprising at least a portion of the impurities and the second cooled process gas stream, wherein the third condenser vessel comprises: a third heat exchanger; a seventh inlet for providing the second process gas stream; an eighth inlet for providing the third cooling medium; a ninth outlet for removal of a third spent cooling medium; a tenth outlet for removal of the second cooled process gas stream; and an eleventh outlet for removal of the third condensate stream; a third condensate tank comprising: a ninth inlet for providing the third condensate stream; a fourth condenser vessel operated under suitable conditions to produce the fourth condensate stream comprising C₂-C₄ alkene (e.g., ethylene and/or propylene) and the second vent gas stream, wherein the fourth condenser vessel comprises: a fourth heat exchanger; a tenth inlet for providing the second cooled process gas stream; an eleventh inlet for providing the fourth cooling medium; a twelfth outlet for removal of a fourth spent cooling medium; a thirteenth outlet for removal of the second vent gas stream; and a fourteenth outlet for removal of the fourth condensate stream; and a fourth condensate tank comprising: a twelfth inlet for providing the fourth condensate stream.

### Embodiment 11

The system of embodiment 10 further comprising a first pre-cooling apparatus for cooling the process gas stream prior to introduction into the first condenser vessel and/or a second pre-cooling apparatus for cooling the process gas stream prior to introduction into the third condenser vessel.

### Embodiment 12

The system of embodiment 10 or 11, wherein the third cooling medium stream is (i) the first pressurized liquid C₂-C₄ alkene stream and/or (ii) liquid and/or gaseous nitrogen.

### Embodiment 13

The system of any one of embodiments 10 to 12, wherein the first cooling medium, the second cooling medium, and/or the fourth cooling medium comprises liquid nitrogen and/or gaseous nitrogen.

### Embodiment 14

The system of any one of embodiments 10 to 13, wherein the first heat exchanger, the second heat exchanger, the third heat exchanger, and/or the fourth heat exchanger are each independently a coil heat exchanger, a shell and tube heat exchanger or a plate heat exchanger.

What is claimed is:

1. A method for recovering C₂-C₄ alkene from a process gas stream comprising:
a first condensation cycle comprising:
cooling the process gas stream comprising C₂-C₄ alkene and impurities in a first condenser vessel with a first cooling medium under suitable conditions to produce a first condensate comprising at least a portion of the impurities and a first cooled process gas stream comprising C₂-C₄ alkene having a temperature of less than or equal to −10° C.;
collecting the first condensate in a first condensate tank;
cooling the first cooled process gas stream comprising C₂-C₄ alkene in a second condenser vessel with a second cooling medium under suitable conditions to produce a second condensate comprising C₂-C₄ alkene and a first vent gas stream; and
collecting the second condensate in a second condensate tank; and
a pressurization cycle comprising:
heating the second condensate tank to produce pressurized liquid C₂-C₄ alkene; and
optionally, recycling at least a portion of the pressurized liquid C₂-C₄ alkene to a second condensation cycle comprising the same steps as the first condensation cycle for use as the first cooling medium.

2. The method of claim 1, wherein the process gas stream enters the first condenser vessel at a temperature of about 40° C. or lower and a pressure of at least about 90 kPa.

3. The method of claim 1, wherein the impurities are nitrogen, hydrogen, water, C₁-C₂₀ hydrocarbons, and/or oxygenates.

4. The method of claim 1, wherein the first vent gas stream comprises nitrogen.

5. The method of claim 1, wherein the first condenser vessel and/or the second condenser vessel each independently comprises a coil heat exchanger, a shell and tube heat exchanger or a plate heat exchanger.

6. The method of claim 1, wherein the first cooling medium and/or the second cooling medium comprises liquid nitrogen and/or gaseous nitrogen.

7. The method of claim 6, wherein the second cooling medium is provided at a temperature of at least about −196° C. and a pressure of less than or equal to about 1500 kPa.

8. The method of claim 1, wherein the pressurized liquid C₂-C₄ alkene is maintained at a pressure of at least about 100 kPa.

9. The method of claim 1, wherein at least about 80 wt % of the C₂-C₄ alkenes present in the process gas stream are present in the second condensate.

10. The method of claim 1, wherein the condensation cycle runs for about 6.0 hours to about 24 hours and the pressurization cycle runs for about 6.0 to about 24 hours.

11. The method of claim 1 further comprising cooling the process gas stream in a pre-cooling apparatus prior to introduction into the first condenser vessel, wherein the process gas stream is cooled to a temperature of about −40° C. or higher.

12. The method of claim 1, wherein the C₂-C₄ alkene is ethylene and/or propylene.

13. A system for recovering C₂-C₄ alkene from a process gas stream comprising:
a first process gas stream comprising C₂-C₄ alkene;
a first cooling medium stream;
a first condensate stream comprising at least a portion of the impurities;
a first cooled process gas stream having a temperature of less than or equal to −10° C.;
a first vent gas stream;
a second cooling medium stream;
a second condensate stream comprising C₂-C₄ alkene;
a second process gas stream comprising C₂-C₄ alkene;
a third cooling medium stream;
a third condensate stream comprising at least a portion of the impurities;
a second cooled process gas stream having a temperature of less than or equal to −10° C.;
a second vent gas stream;
a fourth cooling medium stream;
a fourth condensate stream comprising C₂-C₄ alkene;
a first pressurized liquid C₂-C₄ alkene stream;
a first C₂-C₄ alkene vent gas stream;
a first condenser vessel operated under suitable conditions to produce the first condensate stream comprising at least a portion of the impurities and the first cooled process gas stream, wherein the first condenser vessel comprises:
a first heat exchanger;
a first inlet for providing the first process gas stream;
a second inlet for providing the first cooling medium;
a first outlet for removal of a first spent cooling medium;
a second outlet for removal of the first cooled process gas stream; and
a third outlet for removal of the first condensate stream;
a first condensate tank comprising:
a third inlet for providing the first condensate stream;
a second condenser vessel operated under suitable conditions to produce the second condensate stream comprising C₂-C₄ alkene and the first vent gas stream, wherein the second condenser vessel comprises:
a second heat exchanger;
a fourth inlet for providing the first cooled process gas stream;
a fifth inlet for providing the second cooling medium;
a fourth outlet for removal of a second spent cooling medium;
a fifth outlet for removal of the first vent gas stream; and
a sixth outlet for removal of the second condensate stream;
a second condensate tank comprising:
a sixth inlet for providing the second condensate stream;
a seventh outlet for removal of the first pressurized liquid C₂-C₄ alkene stream; and
an eighth outlet for removal the first C₂-C₄ alkene vent gas stream;
a first means for providing heat to the second condensate tank;
a third condenser vessel operated under suitable conditions to produce the third condensate stream comprising at least a portion of the impurities and the second cooled process gas stream, wherein the third condenser vessel comprises:
a third heat exchanger;
a seventh inlet for providing the second process gas stream;
an eighth inlet for providing the third cooling medium;
a ninth outlet for removal of a third spent cooling medium;
a tenth outlet for removal of the second cooled process gas stream; and
an eleventh outlet for removal of the third condensate stream;
a third condensate tank comprising:
a ninth inlet for providing the third condensate stream;
a fourth condenser vessel operated under suitable conditions to produce the fourth condensate stream comprising C₂-C₄ alkene and the second vent gas stream, wherein the fourth condenser vessel comprises:
a fourth heat exchanger;
a tenth inlet for providing the second cooled process gas stream;
an eleventh inlet for providing the fourth cooling medium;
a twelfth outlet for removal of a fourth spent cooling medium;
a thirteenth outlet for removal of the second vent gas stream; and
a fourteenth outlet for removal of the fourth condensate stream, and a fourth condensate tank comprising:
a twelfth inlet for providing the fourth condensate stream.

14. The system of claim 13 further comprising a first pre-cooling apparatus for cooling the process gas stream prior to introduction into the first condenser vessel and/or a second pre-cooling apparatus for cooling the process gas stream prior to introduction into the third condenser vessel.

15. The system of claim 13, wherein the third cooling medium stream is (i) the first pressurized liquid C₂-C₄ alkene stream and/or (ii) liquid and/or gaseous nitrogen.

16. The system of claim 13, wherein the first cooling medium, the second cooling medium, and/or the fourth cooling medium comprises liquid nitrogen and/or gaseous nitrogen.

17. The system of claim 13, wherein the first heat exchanger, the second heat exchanger, the third heat exchanger, and/or the fourth heat exchanger are each independently a coil heat exchanger, a shell and tube heat exchanger or a plate heat exchanger.

18. The system of claim 13, wherein the C₂-C₄ alkene is ethylene and/or propylene.
